(19) United States
(12) Patent Application Publication
(10) Pub. No.: US 2007/0260319 A1
(43) Pub. Date: Nov. 8, 2007

(54) Testing

(76) Inventor: Christopher Reah, Taunton (GB)

Correspondence Address: JONATHAN SPANGLER;NU VASIVE, INC., 4545 TOWNE CENTRE COURT, SAN DIEGO, CA, 92121, US

(21) Appl. No.: 11/660,719

(22) PCT Filed: Aug. 4, 2005
(86) PCT No.: PCT/GB05/03082

§ 371 Date: Feb. 20, 2007

(30) Foreign Application Priority Data
Aug. 18, 2004 (GB) 0418421.4

(51) Int. Cl. A61F 2/44

(52) U.S. Cl. 623/17.16

(57) ABSTRACT

A method and apparatus for testing an item, such as an artificial and/or prosthetic intervertebral disc or part thereof, is provided to allow one or more combinations of compressive load and/or flexion and/or extension and/or lateral bending and/or axial rotation and/or shear to be considered. The method includes: providing a first structure having an item contacting portion and a second structure having an item contacting portion; providing an item between the first and second structures and in contact with the item contacting portions; operating an actuator to move the first structure between a first state and a second state, the first structure being in a first rotational position relative to the second structure in the first state and being in a different second rotational position relative to the second structure in the second state.

This invention concerns improvements in and relating to testing, particularly, but not exclusively, medical devices such as artificial intervertebral discs.

Some testing devices for medical devices, such as implants exist, with a view to fatigue testing. However, those devices face problems in terms of the speed and/or accuracy and/or expense of testing.

According to a first aspect of the present invention we provide apparatus for testing an item, the apparatus including
a first structure provided with a item contacting portion;
a second structure provided with an item contacting portion;
an actuator for moving the first structure between a first state and a second state;
the first structure being in a first rotational position relative to the second structure in the first state and being in a different second rotational position relative to the second structure in the second state.

According to a second aspect of the present invention we provide a method for testing an item, the method including
providing a first structure having an item contacting portion and a second structure having an item contacting portion;
providing an item between the first and second structures and in contact with the item contacting portions;
operating an actuator to move the first structure between a first state and a second state, the first structure being in a first rotational position relative to the second structure in the first state and being in a different second rotational position relative to the second structure in the second state.

The testing may be to establish the item's performance in response to one or more combinations of compressive load and/or flexion/extension and/or lateral bending and/or axial rotation and/or shear.

Preferably the item is provided between the first and second structures under compression. Preferably the item is under compressive load in the first state and the second state, and more preferably all intermediate states between the first and second state. Preferably the item is under compressive load at all rotational positions of the first structure relative to the second.

Preferably the level of compressive load is different in the first state to the level in the second state. Preferably the level in the first state is different from the level at one or more intermediate states. Preferably the level in the second state is different from the level at one or more intermediate states. Preferably the level at the one or more intermediate states is of a level between that at the first state and at the second state.

The level of compressive load may be the same in the first state as the level in the second state and/or as in one or more intermediate states. Preferably the level of compressive load is the same during the transition from the first state through the one or more intermediate states to the second state and back to the first state.

The level of compressive load may vary sinusoidally, for instance during the transition from the first state through the one or more intermediate states to the second state and back to the first state.

The level of compressive load may vary through a cycle in a predetermined and/or controlled manner.

The level of compressive load may be varied between a first cycle and a second cycle. The first and second cycles may be separated by one or more cycles.

Preferably the variation in compressive load is cyclic. Preferably the cycle starts at the first state, passes through the second state and is completed by a return to the first state.

The cyclic variation in compressive load may be due, wholly or in part, to the cyclic variation in the length of a lever. Preferably the lever supports a mass. The mass may be changed between tests and/or parts of tests. Preferably the length of the lever is the horizontal distance between a vertical line from the centre of the mass and a vertical line extending from the centre of rotation of the first structure.

The compressive load may be, wholly or in part, controlled by an actuator. The actuator may engage with a part of the first structure. The actuator may extend between one part of the first structure and another part of the first structure. The part of the first structure may be a part of a lever. Preferably the length of the lever is the horizontal distance between a vertical line from the centre of the mass and a vertical line extending from the centre of rotation of the first structure. The actuator may extend between a part of the lever and the second link.

The actuator K can be in the form of a pneumatic, hydraulic or other applied force actuator. Preferably the provision of the compressive load is disconnected from the provision of the flexion motion and/or extension motion.

A lever may be included in the first structure. The lever may be or may be part of a link. A link may be included as a part of the first structure. The link may be formed by a first part and a second part. The first part may provide support location for a mass. The second part may be at 90° to the first part, and is preferably substantially vertically provided, at least in one state. The link may be connected to a pivot and is preferably connected to the pivot by the second part. The link may be connected to a first item mount.

The pivot may be formed between the link and a second link. The pivot is preferably part of the first structure. Preferably the second link is part of the first structure. The second link may be formed by a first part and a second part. The first part may provide receive the input from an actuator, preferably a separate actuator to the actuator for the compressive load. The second part may be at 90° to the first part, and is preferably substantially vertically provided, at least in one state. The second link may be connected to a pivot and is preferably connected to the pivot by the second part. The second link may be connected directly to the actuator, preferably a separate actuator to the actuator for the compressive load, or more preferably to a member. The member preferably provides drive from a single actuator, preferably a separate actuator to the actuator for the compressive load, to a plurality of units of the aforementioned type.

The first item mount is preferably part of the first structure. The first item mount may abut the item and/or have the item attached thereto. The first item mount may be provided with one or more removable wedges and/or spacers. The wedge(s) and/or spacer(s) may be used to vary the position of the item relative to the first structure and/or the second structure. The wedge(s) and/or spacer(s) may be used to accommodate different size items and/or different shaped items between the first and second structures. The wedge(s) and/or spacer(s) may be used to vary the Lordosis angle.

The item may be a medical device, particularly an implant. The item may particularly be an artificial and/or prosthetic intervertebral disc or part thereof. A range of different size and/or different shape discs or parts thereof may be tested.

The item may be deformed to a different orientation in the second state to the orientation in the first state. The different orientation may be due to a decrease in the separation of one part of the first structure item contacting portion and the second structure item contacting portion. The different orientation may be due to an increase in the separation of one part of the first structure item contacting portion and the second structure item contacting portion. The different orientation may be due to a decrease in the thickness of a part of the item. The different orientation may be due to an increase in the thickness of a part of the item. Both increases and decreases may occur for different parts.

Preferably the first structure item contacting portion defines a plane and/or the portion of the item contacting the first structure item contacting portion defines a plane. Preferably the second structure item contacting portion defines a further plane and/or the portion of the item contacting the first structure item contacting portion defines a further plane. Preferably the intersection of the plane and further plane define an angle. In one state, preferably the first state, this angle may be equal to the Lordosis angle. Preferably the angle is different in the first state to in the second state. Preferably the angle is greater in the second state. The angle may be zero (that is the planes do not meet) in the first state. Preferably at one or more intermediate states the angle has different intermediate values.

The item may be subjected to a different level of flexion and/or extension and/or forward bending in the first state to the second state. The level may be greater in the second state.

Preferably the variation in deformation and/or variation in angle and/or variation in flexion and/or extension and/or forward bending varies in a cyclic manner. Preferably the cycle starts at the first state, passes through the second state and is completed by a return to the first state.

Preferably the cycles of the compressive load and variation in deformation and/or variation in angle and/or variation in flexion and/or extension and/or forward bending coincide. Preferably coincidence between the cycles is provided due to the length of the lever for the compressive load varying in the same manner as the other variation and/or due to the actuator controlling compressive load varying in the same manner as the other variation.

Preferably the variation in deformation and/or variation in angle and/or variation in flexion and/or extension and/or forward bending is caused by the actuator, preferably a separate actuator to the actuator for the compressive load. Preferably it is caused by the downward movement of the second link and/or movement of the second link towards the second structure. Preferably it is caused by the end of the link which provides the pivot moving towards the second structure. Preferably it is caused by the end of the link which supports the mass moving away from the second structure. Preferably the end of the link which provides the pivot is closer to the second structure in the second state than in the first state. Preferably the end of the link which supports the mass is closer to the second structure in the first state than in the second state.

The variation in deformation and/or variation in angle and/or variation in flexion and/or extension and/or forward bending may arise due to movement

The transition from first state to second state may involve a rotation of greater than 0° to 45°, preferably greater than 0° to 25°. The actuator, preferably a separate actuator to the actuator for the compressive load, may drive the transition from first state to second state and/or second state to first state.

The second item contacting portion may be provided by a second item mount, preferably one that is part of the second structure. The second item mount may abut the item and/or have the item attached thereto. The second item mount may be provided with one or more removable wedges and/or spacers. The wedge(s) and/or spacer(s) may be used to vary the position of the item relative to the second structure and/or the first structure. The wedge(s) and/or spacer(s) may be used to accommodate different size items and/or different shaped items between the first and second structures. The wedge(s) and/or spacer(s) may be used to vary the Lordosis angle.

The second item mount may be slidable mounted relative to a supporting structure. The second item mount may be provided on a block which is slidable mounted relative to the supporting structure. The slidable mounting may be provided by ball bearings at the interface between the block and the support structure. The block may move in a linear manner, particularly during the transition from first state to second state. Preferably the block reciprocates.

A bearing may be provided to allow rotational movement of the second item mount. Preferably the bearing is provided between the block and the second item mount. Preferably the bearing allows rotational movement of the second item mount relative to the block. Preferably the axis of rotation is perpendicular to the axis of sliding of the block.

The second item mount may have a first position relative to the first item mount in the first state and a second position relative to the first item mount in the second state. Preferably the transition from first to second position involves lateral movement of the second item mount relative to the first item mount. Preferably the transition from first position to second position applies a shear load on the item.

Preferably the second item mount is connected to a further block. Preferably the further block is fixed relative to a support structure. The connection may be provided by an element, such as a bar. Preferably the connection with the further block guides the movement of the second item mount during the transition from first state to second state and/or second state to first state. Preferably the connection involves an engagement with a guide way on or in the further block. The guide way may take the form of a groove and/or channel and/or slot. Preferably the connection is free to slide within the guide way. Preferably the guide way is at an angle relative to the motion of the block and/or the axis of rotation of the first structure.

Preferably in the first state the connection is in a part of the guide way nearer the second item mount than the part it occupies in the second state. Preferably the connection occupies intermediate parts during the transition from first to second state.

Preferably the second item mount rotates during the transition from first to second state and/or during the transition from second to first state. Preferably the rotation of the second item mount applies an axial rotational load to the item. Preferably the rotation provides an axial rotational load at the same time as a variation in deformation and/or variation in angle and/or variation in flexion and/or extension and/or forward bending. The rotation of the second item mount and the rotation of the first structure relative to the first may occur at the same time, and preferably occurs at the same time and gives rise to lateral loading of the item and/or lateral bending.

Preferably the axial loading varies in a cyclic manner. Preferably the cycle starts at the first state, passes through the second state and is completed by a return to the first state.

Preferably the cycles of the axial loading and variation in deformation and/or variation in angle and/or variation in flexion and/or extension and/or forward bending coincide.

The apparatus and/or method may provide for adjustable levels of compressive load and/or flexion and/or axial load and/or lateral load and/or shear to be applied.

A plurality of pairs of first and second structure may be provided. Preferably all pairs are powered by a common actuator, preferably a separate actuator to the actuator for the compressive load. The same actuator may be used for the compressive load of a plurality of pairs of first and second structure. Different pairs may be used to test different size and/or shape items during a single test run.

Various embodiments of the invention will now be described, by way of example only, and with reference to the accompanying drawings in which:—

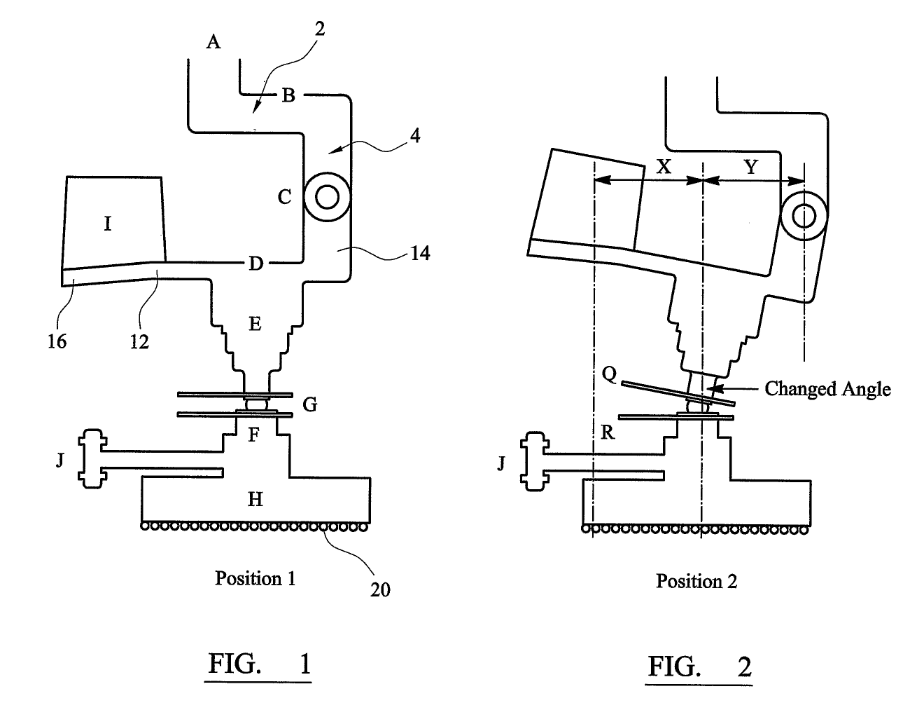

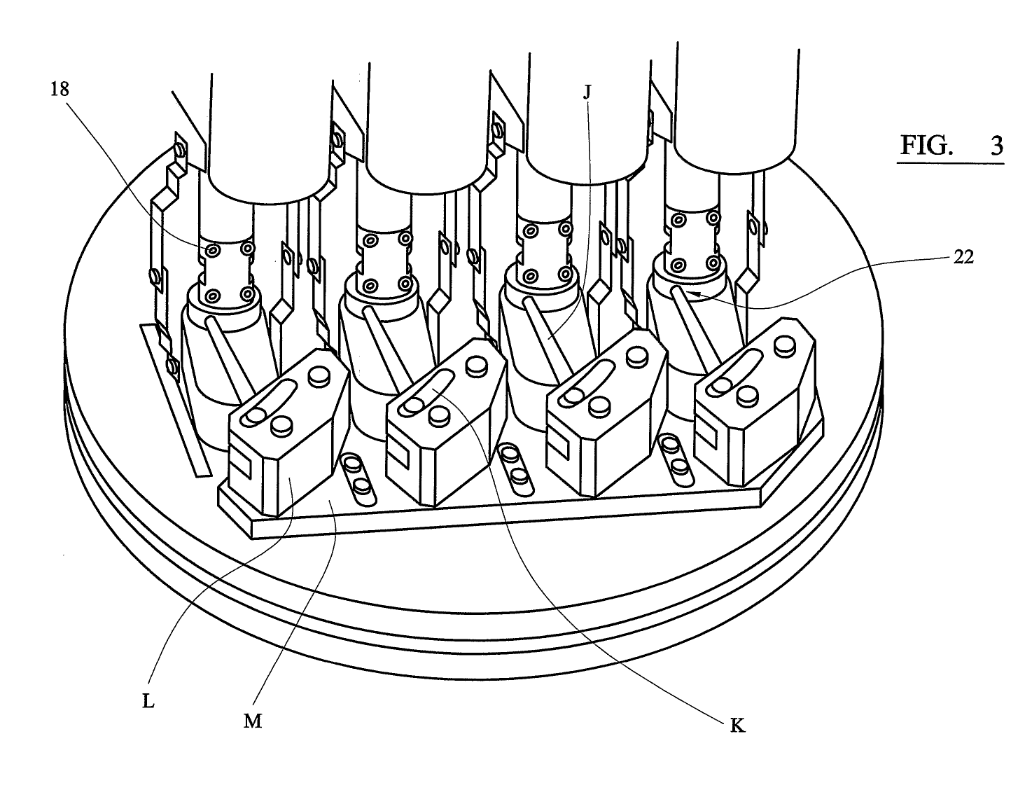

FIG. 1 is a schematic side view of a test rig according to the invention in a first state;

FIG. 2 is a schematic side view of the test rig of FIG. 1 in a second state;

FIG. 3 is a photography of a test rig according to the present invention;

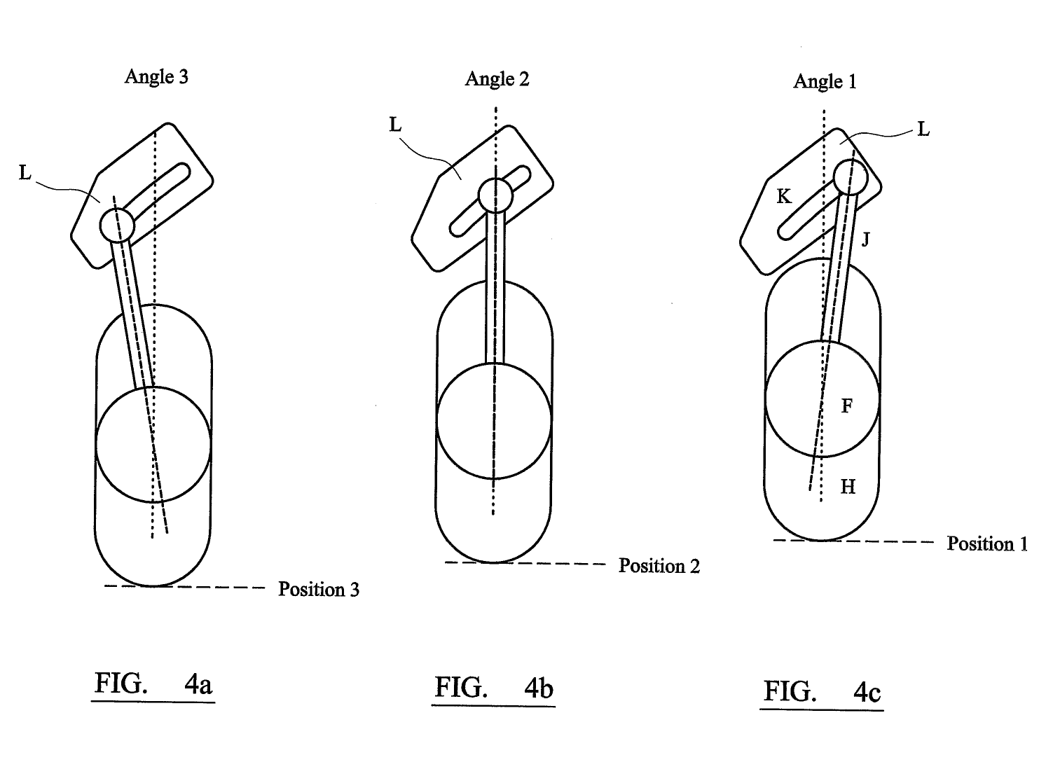

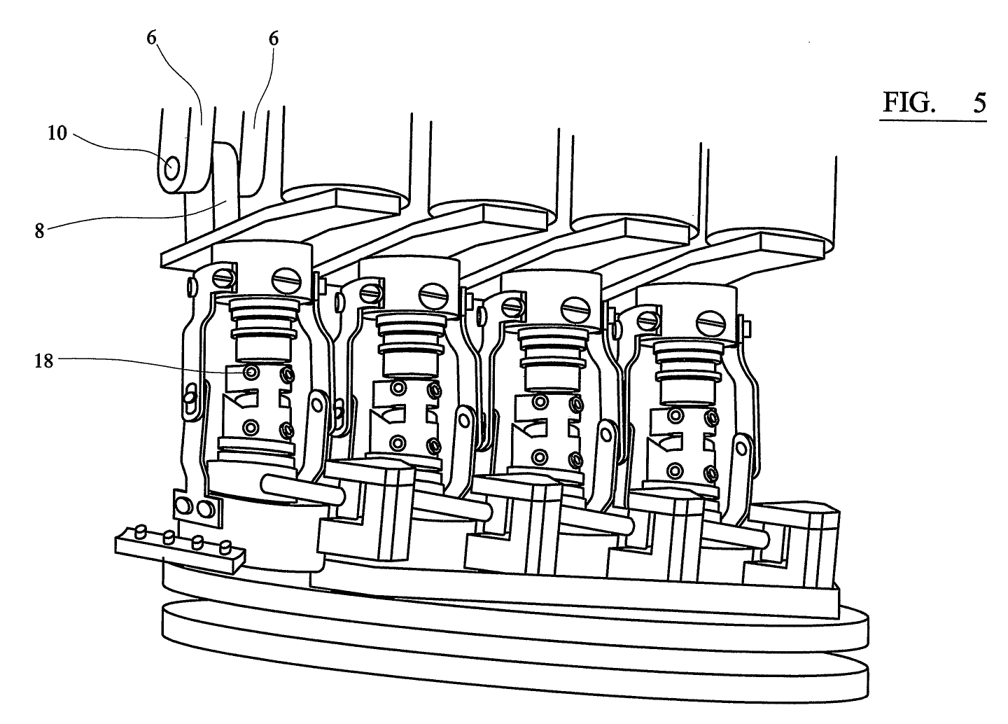

FIG. 4a is a schematic plan view of the lower structure of the test rig of FIG. 1;

FIG. 4b is a schematic plan view in a state intermediate to those of FIGS. 4a and 4c;

FIG. 4c is a schematic plan view of the lower structure of the test rig of FIG. 2;

FIG. 5 is a further photography of a test rig according to the present invention;

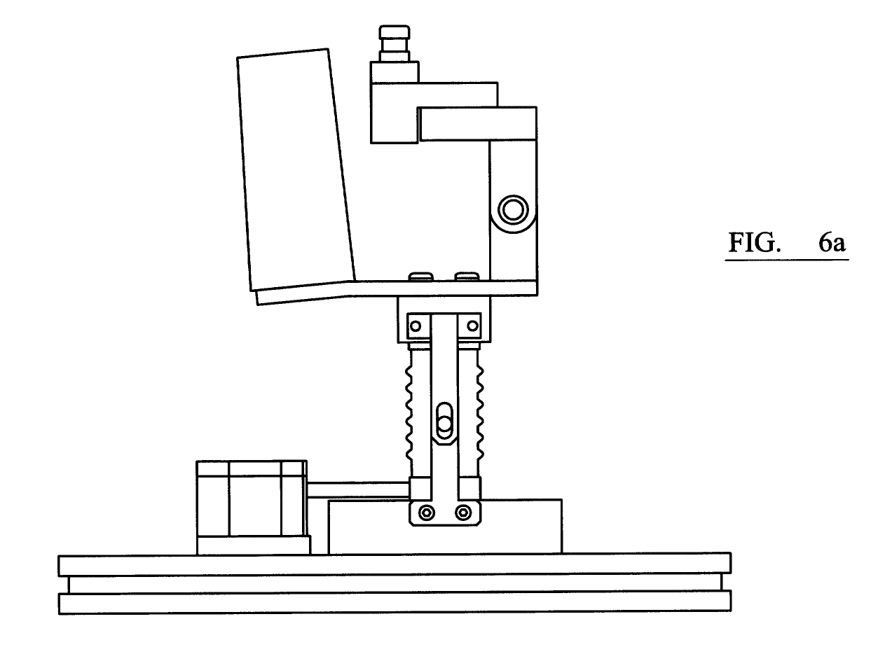

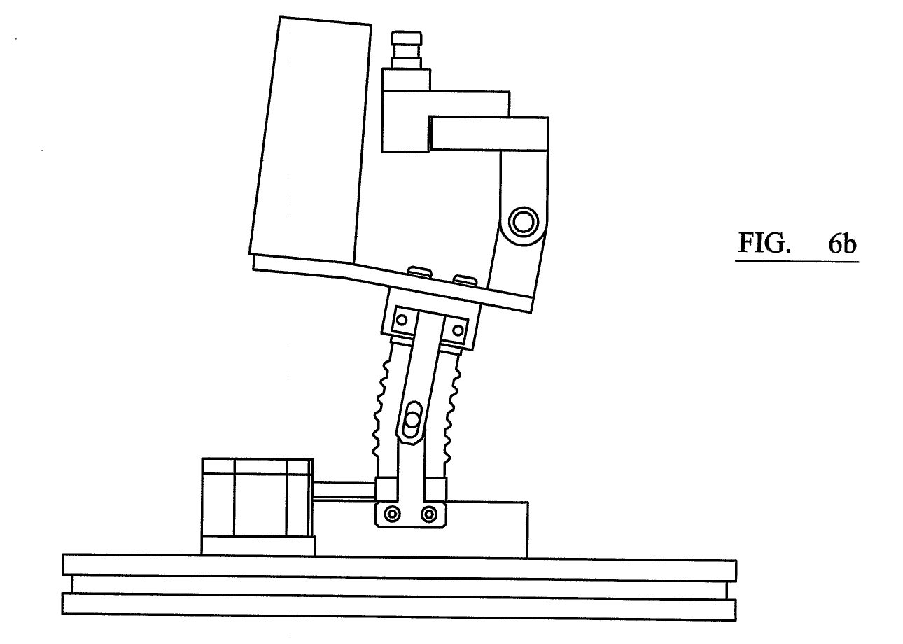

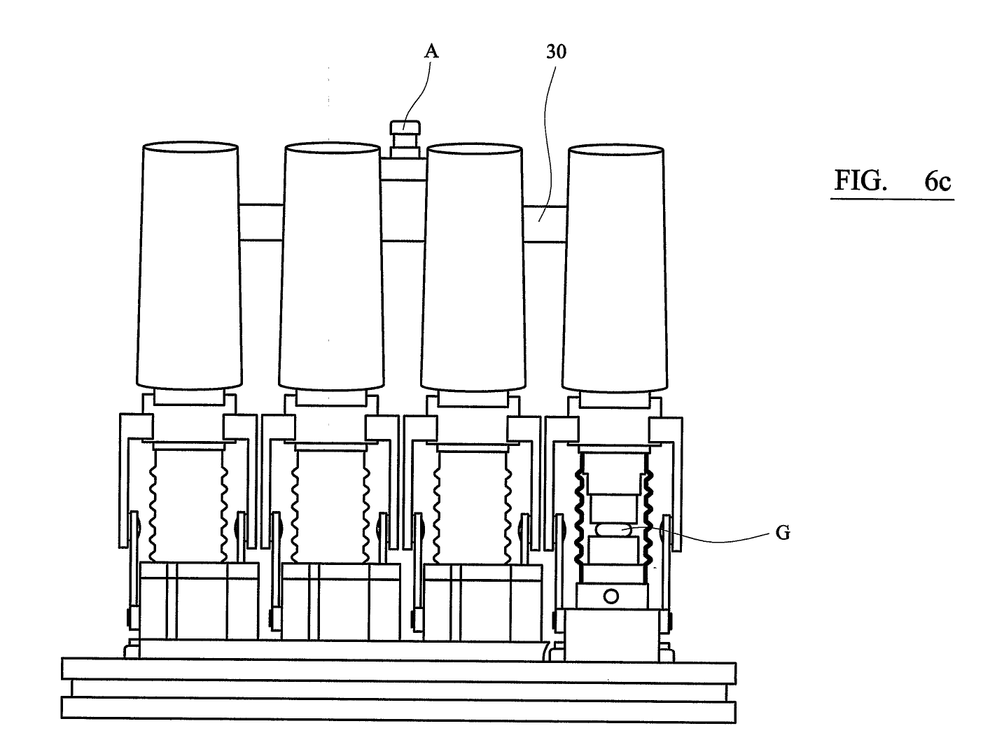

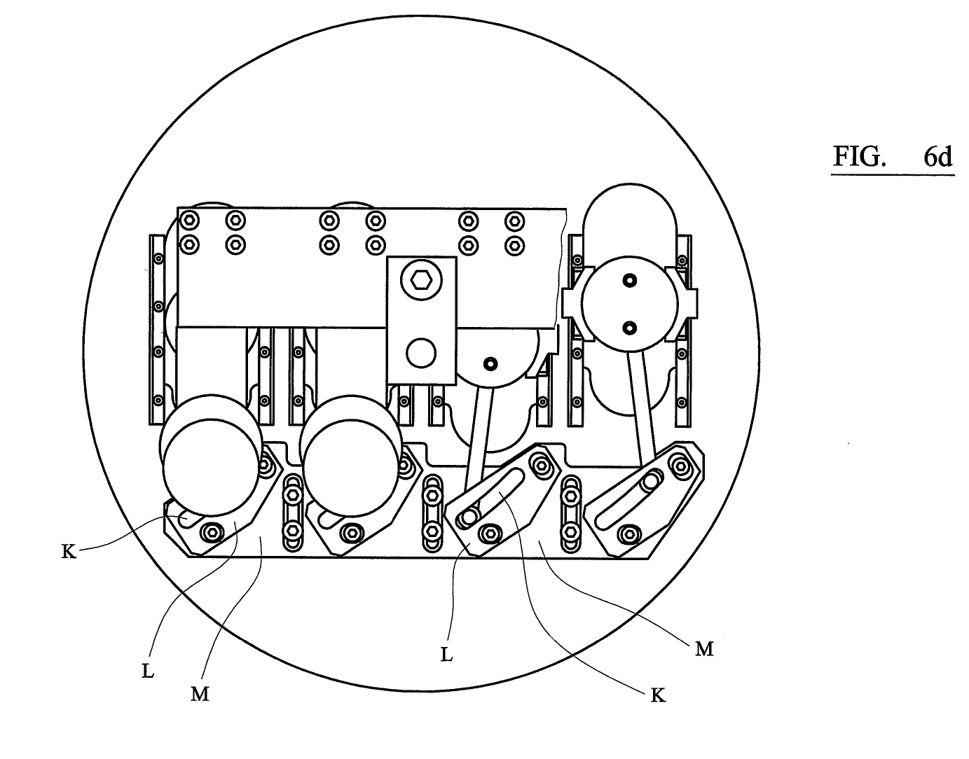

FIGS. 6a, 6b, 6c and 6d provide views of a test rig according to the present invention; and

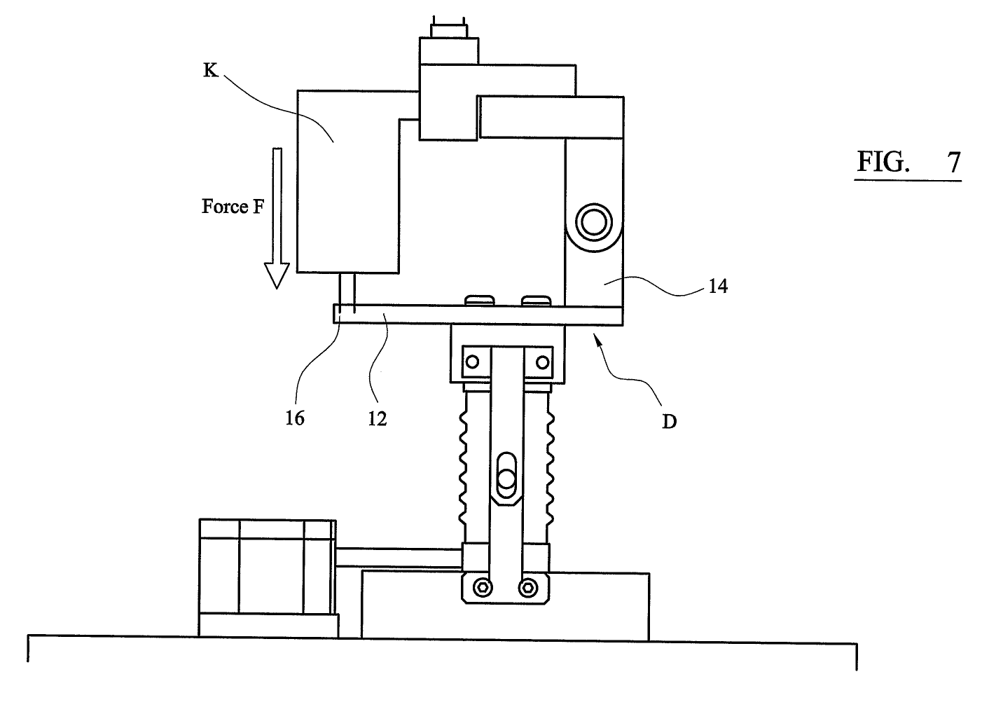

FIG. 7 is a schematic side view of a test rig according to another embodiment of the invention in a first state.

Artificial intervertebral discs are a large growth area in the medical industry. As part of the development of implants extensive fatigue testing is required. With fatigue testing required, but equipment for it not available, each organisation thus far has had to define its own testing methods and produce its own testing equipment. This makes comparison between sets of test results difficult. The approaches taken in such designs have also give rise to other problems or limitations on the testing.

The applicant has therefore sought to provide improved equipment and methods for fatigue testing. The result is a test rig which accurately provides the type and characteristics of the forces encountered in a spine. In the human spine there is a variable compressive force acting at all times. Additionally the main motions and loads that the spine undergoes are:

Flexion/Extension (forward bending),

Lateral Bending (bending to the side),

Axial Rotation (twisting) and

Shear (relative horizontal movements)

All of these are replicated by the applicant's test rig. A compressive load that mimics the physiological situations encountered is combined with a flexion/extension load and the option of a simultaneous coupled axial rotation load. In addition the coupling of the flexion/extension load with the axial rotation load will induce a simultaneous small lateral bending load. This is a substantial advance over existing test rigs which have been limited in the extent to which they truly test the type of fatigue of interest. Generally speaking, only two loads types are considered at a time. These are usually compression and one other. The manner in which the loads are considered may also not reflect the real world characteristics of such loads. For instance, the compression load may be applied simultaneously with the other load, and as a result the compression load pattern will not resemble the physiological pattern it is intended to replicate.

The test rig provided by the applicant's is also beneficial in being able to test many implants at once using a single test rig. This is a significant benefit as a substantial amount of testing needs to be performed on implants. Furthermore, many material used (such as polymers) must be tested at low frequencies such as 1 Hz and a typical fatigue test for an artificial disc must last for 10 million cycles. Some fatigue tests may take months to complete, therefore. To get through a suitable number of test samples in a given time period, some companies have relied upon a bank of fatigue test machines to increase their testing rates. The number of separate machines required makes such banks extremely expensive to provide. There may also be issues of the testing not being identical across the separate machines. The applicant has provided multiple testing on a single test rig, however, without taking the approach of providing multiple actuators on the test rig. Multiple actuators would be one potential solution to the problem. However, the result would be complex machinery that has a series of actuators to apply loads in each of the individual directions. Such systems would be expensive and would be difficult to programme, difficult to validate once programmed and difficult to apply to more than one test specimen at a time.

As well as testing multiple implants on a single test rig, therefore, the applicant has designed a test rig which does so using a single actuator.

These developments provide a highly attractive test rig which makes it possible for standardised testing methods and equipment to be used to compare different implants with one another. The developments also mean that international standards bodies are assisted in investigating the issue of test protocols and the test methods used to enact these protocols. Better testing will also give improvements in patient safety and the assurance level which can be provided to patients. The complex loading found in the spine makes testing requirements both difficult to predict and difficult to model.

Referring to FIG. 1, a schematic side view of one unit of the test rig is shown in a first state. FIG. 2 shows a schematic side view of the same test rig as FIG. 1, but in a different state.

The drive for the movement arises from an actuator A which is connected to link B. The link B is in the form of a first part 2 and second part 4, provided at 90° to the first. The first and second parts are fixed in their orientation to one another. The first part extends in a generally horizontal direction and the second extends in a generally vertical direction. Movement of the actuator causes the link B to move downward, relative to the Figure, from the position of FIG. 1 to the position of FIG. 2. The actuator provides a reciprocating motion, and further motion returns the link B to the position of FIG. 1. The frequency of reciprocation can be varied, for instance using control electronics.

A pivot C is provided. This is formed by the twin spaced spokes 6 of link B receiving into that space a single spoke 8 provided on the end of link D. A pin 10 passes through coaxial holes in the twin spokes of link B and spoke of link D to form the pivot C. The pivot C facilitates rotation of link D relative to link B during the transition from the first stage illustrated in FIG. 1 to the second stage illustrated in FIG. 2, and back again.

The link D is in the form of a first part 12 and second part 14, provided at 90° to the first. The first part extends in a generally horizontal direction and the second extends in a generally vertical direction. The first and second parts are fixed in their orientation to one another. The second part provides at its end the spoke which forms part of the pivot C. The first part has a substantial extent and at the end 16 distal to the second part provides a support location for mass I. The mass I can be varied as desired. During the transition from the first state of FIG. 1 to the second state of FIG. 2, downward displacement of the link D is resisted by the remainder of the lower structure discussed below. The flexibility offered by the implant G and/or the movement of the lower part of the structure discussed below, however, means that the second part 14 of link D and that end of link D can move downward, with the other end 16 of link D and, as a result, the mass I moving upward. The transition back to the first state returns link D to a level position. The mass I and/or the length of the lever provided by link D can be changed to varying the test conditions applied between tests.

The bottom of the link D is rigidly connected to an upper mount E. The upper mount E and lower mount F receive between them an implant G to be tested. The implant G may be attached to the upper mount E and lower mount F, for instance by the pins 18 shown in FIG. 5. Alternatively, the implant G may be retained by the compressive load exerted upon it and/or by the shape of the upper mount E and/or lower mount F. As the transition from the state of FIG. 1 to the state of FIG. 2 occurs, the rotation of link D and hence upper mount E, causes deformation in the implant G. A change in angle between the upper and lower mount surfaces and/or planes defined by the upper and lower mount and/or the upper and lower surfaces of the implant and/or planes defined by the implant (lines Q and R) occurs. As a result, the flexion/extension motion is applied to the implant.

The lower mount F is positioned on a block H which is capable of sliding motion relative to the support J below it due to ball bearings 20. As a consequence, both the lower mount F and block H are capable of reciprocating motion. During the transition from the first state of FIG. 1 to the second state of FIG. 2, the loads transferred through the implant cause the lower mount and hence block H to move from right to left, relative to the drawings. There is potential to apply shear load to the implant in this way.

It should also be noted that the lower mount F is connected to a block L rigidly attached to support M. The connection is provided by a bar J which is provided, at the end distal to mount F, with a cross bar. The cross bar is received within a groove K in the block L and is free to move within the constraints of that groove K. The groove K is angled with respect to the reciprocating motion of lower mount F and block H. As a result of this structure, the reciprocating motion imparted to lower mount F and block H by the rotation of link D and upper mount E causes the bar J to move relative to block L. However, this movement is constrained by the groove K and can only occur through the bar J deviating in its motion from the axis of the reciprocation of lower mount F and block H (fixed dotted line in FIGS. 4a, 4b, 4c). This deviation imparts a rotation to lower mount F relative to block H and relative to the upper structure too. The rotation is facilitated by a bearing 22, shown in FIG. 3. As a result of this configuration, axial rotation or twisting too is applied to the implant G.

The variation in position can be seen in FIGS. 4a, 4b and 4c. The position of FIG. 4a generally corresponds to the position in the first state of FIG. 1. As the block H slides towards the block L so the bar J moves in the groove K and the angle changes. This transition occurs through the position 2 situation of FIG. 4b, to the position of FIG. 4c which generally corresponds to the second state position of FIG. 2. The angular extent of the rotation can be varied by altering the path that the track takes.

Throughout the testing, the upper structure can of course be used to apply a compressive load of the desired level to the implant G. Significantly in the structure of the present invention, the effective leverage of the mass I changes as the position of the block H changes through sliding and this has the effect of oscillating the compressive load in a manner more representative of real world conditions.

A series of such units can be provided alongside one another, see FIG. 3 and FIG. 5, to provide simultaneous testing of multiple implants. A single actuator applies its motion to a cross bar which spans all units and feeds the motion to link B in each case.

FIG. 6a illustrates a side view of a test rig according to the invention, once again in the first state. FIG. 6b illustrates that test rig in the second state. FIG. 6c is a front view of the test rig of FIGS. 6a and 6b and clearly shows the four separate units provided, the common actuator A and the common bar 30 for communicating the motion of the actuator to the link B in each case. FIG. 6d is a plan view of the same test rig and shows the support M, block L and groove K.

In the alternative embodiment of FIG. 7, once again the link D is in the form of a first part 12 and second part 14, provided at 90° to the first. The first part 12 has a substantial extent and at the end 16 distal to the second part 14 provides an engagement location for actuator K. In this embodiment the actuator K replaces the variable mass I. can be varied as desired.

During the transition from the first state to the second state, of the type illustrated above in FIGS. 1 and 2, downward displacement of the link D is resisted by the remainder of the lower structure. The flexibility offered by the implant G and/or the movement of the lower part of the structure, however, means that the second part 14 of link D and that end of link D can move downward, with the other end 16 of link D moving upwards against the actuator K.

By using an actuator K instead of a mass, it is possible to vary the compressive load more readily and/or to vary the compressive load in more complex manners. The actuator K can be in the form of a pneumatic, hydraulic or other applied force actuator. This arrangement allows the disconnection of the compressive load provision from the flexion and extension motions. The actuator K thus rends the compression as a totally independent variable. The actuator K can be used to apply different compressive loads, for instance at different times during the testing. The actuator K can also be used to provide more complex compressive load patterns than a simple mass I. For instance, a constant compressive load can be applied throughout a cycle of the jig's movement. Sinusoidal or other load patterns can equally well be applied through appropriate control of the actuator K.

In summary, these designs of test rig can solve the problems in the time taken for each test, the accurate application of all the combined loads and the provision of a compression load which behaves consistently with quite differently to the normal physiological loads. The invention has the advantages of:—
1. The ability to provide a compressive load that oscillates within physiological limits (never goes to zero) combined with a flexion/extension movement/load
2. The ability to couple an axial rotation load with the flexion/extension load so that they are in phase—the magnitude of each load can be adjusted in relation to each other
3. The modular design of the fatigue test jigging such that multiple implants can be simultaneously subjected to loads in multiple axes from a single actuator on the fatigue test machine. Thus, only one fatigue test machine is required to simultaneously test a number of implants
4. The modular design of the fatigue test jigging also allows one fatigue test machine to simultaneously test a number of similar of different sized implants
5. The discs can start their testing with a range of potential lordosis angles (the lordosis angle is the natural flexion/extension angle—usually though of when the person is stood upright with head angled horizontally—it is different for every person and at different levels of the spine). Often the lordosis angle will be built into a jig or changed by a series of wedges or spacers.

Various sized discs can be accommodated through the use of spacers or smaller fixtures to keep all discs with the same lordosis angle relative to each other.

1. A method for testing a medical implant, the method including:
providing at least one implant mount, the implant mount including a first structure having a first implant contacting portion and a second structure having a second implant contacting portion;
providing a medical implant between the first and second structures such that the implant is in contact with the first and second implant contacting portions; and
operating an actuator to move the first structure between a first state and a second state, the first structure being in a first rotational position relative to the second structure while in the first state and a second rotational position relative to the second structure while in the second state.

2. The method of claim 1, wherein the testing establishes a performance of the medical implant in response to at least one of compressive load, flexion, extension, lateral bending, axial rotation, and shear, and any combination of compressive load, flexion, extension, lateral bending, axial rotation, and shear.

3. (canceled)

4. The method of claim 1, wherein the implant is at least a part of at least one of an artificial and prosthetic intervertebral disc.

5. The method of claim 1, wherein the implant is provided between the first and second structures under compression.

6. The method of claim 5, wherein the level of compressive load in the first state is different from the level of compressive load in the second state.

7. (canceled)

8. The method of claim 6, wherein the level of compressive load varies between a first cycle and a second cycle.

9. The method of claim 8, wherein cyclic variation in compressive load is due at least in part to the cyclic variation in at least one of the length of a lever and the force applied by an actuator.

10. (canceled)

11. The method of claim 1, wherein at least one of the first implant contacting portion and the portion of the implant contacting the first implant contacting portion defines a first plane, at least one of the second implant contacting portion and the portion of the implant contacting the second implant contacting portion defines a second plane, the intersection of the first plane and the second plane defines an angle, and the angle is different in the first state as to in the second state.

12-21. (canceled)

22. An apparatus for testing a medical implant, the apparatus comprising:
a first structure provided with a first implant contacting portion;
a second structure provided with a second implant contacting portion;
an actuator for moving the first structure between a first state and a second state;
the first structure being in a first rotational position relative to the second structure while in the first state and being in a different second rotational position relative to the second structure while in the second state.

23. (canceled)

24. The apparatus of claim 23, wherein the medical implant is at least one of an artificial disc, prosthetic intervertebral disc, and any part thereof.

25. (canceled)

26. (canceled)

27. The apparatus of claim 22, wherein the implant is subjected to variation in compressive load and the variation in compressive load is, wholly or in part, controlled by at least one of force applied by an actuator and a length of a lever.

28. The apparatus of claim 27, wherein the actuator extends between a first part of the first structure and a second part of the first structure.

29. The apparatus of claim 27, wherein the actuator is in the form of at least one of a pneumatic, hydraulic and other applied force actuator.

30-35. (canceled)

36. The apparatus of claim 22, wherein the second implant contacting portion is provided with at least one removable spacer.

37-41. (canceled)

42. An apparatus for testing a medical implant, the apparatus comprising:
a first implant mount dimensioned to impose a first compressive force upon a first medical implant;
a second implant mount dimensioned to impose a second compressive force upon a second medical implant; and
an actuator coupled to said first and second implant mounts, said actuator configured to vary the first and second compressive loads such that said first and second implants are each moved from a first position to a second position.

43. The apparatus of claim 42, wherein the testing establishes a performance of the medical implant in response to at least one of compressive load, flexion, extension, lateral bending, axial rotation, and shear, and any combination of compressive load, flexion, extension, lateral bending, axial rotation, and shear.

44. The apparatus of claim 22, wherein the testing establishes a performance of the medical implant in response to at least one of compressive load, flexion, extension, lateral bending, axial rotation, and shear, and any combination of compressive load, flexion, extension, lateral bending axial rotation, and shear.
